(19) United States
(12) Patent Application Publication
(10) Pub. No.: US 2005/0260189 A1
(43) Pub. Date: Nov. 24, 2005

(54) Microbubble compositions, and methods for preparing and using same

(76) Inventors: Alexander L. Klibanov, Charlottesville, VA (US); Klaus F. Ley, Charlottesville, VA (US); Joshua J. Rychak, Charlottesville, VA (US)

Correspondence Address: WOODARD, EMHARDT, MORIARTY, MCNETT & HENRY LLP, BANK ONE CENTER/TOWER, 111 MONUMENT CIRCLE, SUITE 3700, INDIANAPOLIS, IN, 46204-5137, US

(21) Appl. No.: 10/511,383

(22) PCT Filed: Jul. 11, 2003
(86) PCT No.: PCT/US03/21712

§ 371 Date: May 18, 2005

Related U.S. Application Data
(60) Provisional application No. 60/395,179, filed on Jul. 11, 2002.

(51) Int. Cl. A61K 39/395

(52) U.S. Cl. 424/130.1

(57) ABSTRACT

Described are microbubble compositions including microbubbles having membranes that incorporate modified surface features that may be useful, for example, in facilitating binding to a target surface or substance. The surface features may include non-spherical attributes such as crenations, folds, projections, or wrinkles, which can increase the deformability of the microbubble membrane. Such microbubble compositions can be incorporated into targeted ultrasound contrast agents and methodologies. Methods for preparing modified microbubble compositions include providing microbubbles having spherical membranes, and converting the spherical membranes to non-spherical membranes having surface features as mentioned above. Targeting substances can be incorporated into the membranes before or after their conversion from spherical to non-spherical forms.

### REFERENCE TO RELATED APPLICATION

The present application claims the benefit of U.S. Provisional Patent Application Ser. No. 60/395,179 filed Jul. 11, 2002, which is hereby incorporated by reference herein in its entirety.

### GOVERNMENT RIGHTS

This invention was made with support from government grant numbers NIH T32 HL07284-26 and RO1 HL64381. The government has certain rights in the invention.

### BACKGROUND

The present invention relates generally to microbubble compositions and methods, and in particular to microbubble compositions targeted to bind to specific substrates or substances.

As further background, gas filled microbubbles in a region under ultrasonic investigation significantly improve the contrast of grey-scale and Doppler ultrasound. This contrast enhancement is due to an acoustic impedance mismatch between the microbubbles' encapsulated gas and the surrounding blood. The acoustic pulse emitted by the ultrasound transducer is reflected back (backscattered) at the interface between materials with different impedances; the larger the impedance mismatch, the greater the proportion of ultrasound backscatter, and the greater ultrasound contrast achieved. When an ultrasonic acoustic pulse hits a microbubble, it induces volumetric oscillations of the bubble's gaseous phase. These oscillations are highly non-linear and result in significant backscatter, which can be detected by the transducer array (Forsberg and Shi, 2001). The backscatter produced by microbubble oscillations may be several orders of magnitude greater than that produced by the tissue structures under investigation (Klibanov, 1999).

The use of microbubbles as ultrasound contrast agents (UCA's) has many potential clinical applications. These include visualization of cardiac anatomy (Shub et. al, 1976; Crouse et al, 1993), estimation of organ or tissue perfusion (Rim et al, 2001; Mulvagh et al, 2000), and assessment of myocardial viability (Villanueva 2000). Each of these applications utilizes freely flowing microbubbles; that is, the microbubbles do not necessarily remain adherent to the tissue under investigation. Although freely flowing UCA's are able to provide clinically relevant information at the tissue level, inducing microbubbles to bind to a region of interest may enable ultrasound imaging on the molecular scale. Microbubbles may be targeted to specific molecules by affixing a targeting molecule to the outer surface of the bubble. This allows very spatially localized detection of pathology in a tissue under investigation, in addition to the possibility of delivering bioactive substances to said tissue.

There are many factors that influence the efficacy of microbubble contrast agents. Clinical doses of intravenous UCA's for myocardial imaging are on the order of 10⁹ microbubbles (Gunda and Mulvagh, 2001). However, Klibanov et al (1997) showed that a dose of as little as 20 microbubbles per mm3 of blood is sufficient for left-ventricular opacification, and that as little as 3% coverage on a flat surface produces detectable ultrasound signal, using fundamental (not harmonic) imaging. These results suggest that clinical ultrasound visualization may be achieved with low quantities of microbubbles; however, the degree of ultrasound contrast increases with microbubble concentration at the target site. As would be expected, there is an upper limit on the quantity of UCA's that may safely be injected into the body without causing capillary obstruction. Thus, an optimal balance of safety and imaging resolution may be achieved by injecting a small number of precisely-targeted contrast microbubbles.

There is limited prior art pertaining to targeted microbubble compositions. Early observations of lipid and protein shell microbubbles revealed preferential accumulation in the liver (Girard et al, 2001), spleen and lungs (Walday et al, 1994), and reperfused myocardium (Keller et al, 1990; Villanueva et al, 1997). This phenomenon was subsequently revealed to be due to microbubble entrapment in regions of stagnent flow, for example in the liver sinusoids (Kono et al, 2001), and phagocytosis of microbubbles by activated leukocytes (Lindner et al, 2000). Recent publications have shown the feasibility of imaging inflammation by targeting microbubbles to ICAM-1 (Villanueva et al, 1997; Weller et al, 2003), P-selectin (Lindner et al, 2001), and activated leukocytes (Lindner et al, 2000; Christiansen et al, 2002) by altering the microbubble shell composition or immobilizing antibodies on the microbubble surface. Angiogensis, specifically in the case of tumor, has been investigated with microbubbles targeted to the integrin αᵥβ₃ (Leong-Poi et al, 2003; Ellegala et al, 2003), and thrombus has been targeted by microbubbles binding the platelet receptor GPIIb/IIIa (Schumann et al, 2003). Additionally, targeted microbubbles have been described for the purpose of gene (Teupe et al, 2002; Porter et al 2001) and drug (Price et al, 1998) delivery.

Formulations for targeted microbubbles have been described prior to the above mentioned publications. For example, U.S. Pat. No. 6,264,917 describes the formulation of diagnostic contrast agents consisting of a reporter moiety capable of interacting with ultrasound, a targeting vector having affinity to the target site, and a linker connecting the vector to the reporter. U.S. Pat. No. 6,245,318 discloses a microbubble structure capable of mediating selective binding to the target site by immobilizing the targeting vector upon a polymeric spacer arm. The incorporation of more than one targeting vector for the purpose of enhancing adhesion to the target substrate is described in U.S. Pat. No. 6,331,289. Targeted microbubbles have also been described implicitly in bioactive delivery schemes, for example in U.S. Pat. No. 6,443,898. Utilizing the destruction of microbubbles to deliver a bioactive substance into a targeted tissue was described in the acoustically active drug delivery system of U.S. Pat. No. 6,416,740.

Careful study of the published prior art reveals that, although several of the targeted microbubble schemes described above are able to achieve microbubble accumulation at the target site both in vitro and in vivo, there is a high level of non-specific adhesion in each of these formulations. For example, lipid shell microbubbles targeted to vascular inflammatory proteins show adhesion under non-inflamed control conditions of approximately 0.2 (Weller et al, 2003) to 0.3 (Lindner et al, 2001) times that of inflamed tissues both in vitro and in vivo. Although the choice of the targeting molecule partially dictates the microbubble: target binding affinity, the topographical surface features and the mechanical structure of the microbubble is critical to this process. The biophysics of attaching a free-flowing particle to a flat substrate have been studied in depth, with respect to, for example, leukocyte adhesion to the vascular endothelium. The earliest intravital observations of leukocyte adhesion reported spherical leukocytes becoming deformable and assuming a teardrop-shaped profile upon attachment to the endothelium (Atherton and Born, 1972; Firrell and Lipowsky, 1989), and recent studies have confirmed that leukocyte topography (Finger et al, 1996) and deformability (Park et al, 2002; Yago et al, 2002) are critical to the process of leukocyte adhesion. In a manner similar to the described biological process, the attachment of intravascular microbubbles to a target site may be enhanced by tailoring the microbubble structure to achieve a more efficient mechanism of adhesion. The current invention describes the composition and method of preparation and use of an alternative scheme for targeting therapeutic and/or diagnostic microbubbles.

### SUMMARY OF THE INVENTION

Accordingly, in one aspect, the present invention provides microbubble compositions having microbubbles with non-spherical membranes and exhibiting an increased capacity to bind to a targeted surface or substance as compared to corresponding spherical microbubbles. In one embodiment, the invention provides a microbubble composition for binding to a target, comprising gas-filled microbubbles in a liquid carrier. A substantial percentage (i.e. at least about 20 percent) of the microbubbles in the composition have crenated microbubble membranes, wherein the membranes also include targeting molecules that bind to the target.

In another embodiment, the invention provides a microbubble composition useful for binding to a target, comprising a suspension of gas-filled microbubbles in a liquid carrier, wherein a substantial percentage of the microbubbles have membranes including surface projections and targeting molecules that bind to the target.

In another embodiment, the invention provides a microbubble composition useful for binding to a target, comprising a suspension of microbubbles in a liquid carrier, a substantial percentage of the microbubbles having non-spherical microbubble membranes possessing excess surface area with respect to the area of the gas core. The non-spherical microbubble membranes include a targeting molecule for binding to the target and exhibit an increased deformability under fluid shear stress relative to corresponding spherical microbubble membranes.

Another embodiment of the invention concerns an ultrasound contrast agent, comprising a microbubble composition having membranes that include crenations, projections, or non-spherical features as described herein, and a pharmaceutically acceptable liquid carrier. Ultrasound imaging methods using such contrast agents are also provided.

Another embodiment of the present invention concerns a method for preparing a targeted microbubble composition. The method includes forming gas-filled microbubbles having spherical microbubble membranes suspended in a liquid carrier, converting the spherical microbubble membranes to non-spherical microbubble membranes, and incorporating into the microbubble membranes targeting molecules for binding to a target.

Other embodiments of the invention include therapeutic, diagnostic and extraction methods that utilize microbubble compositions of the present invention.

Additional embodiments as well as features and advantages of the invention will be apparent from the descriptions herein.

### DESCRIPTION OF THE FIGURES

FIG. 1 provides a diagram of the inverted parallel plate flow chamber used to perform the in vitro attachment efficiency studies. The target substrate (P-selectin.Fc) is adsorbed onto the top plate of the flow chamber, and a dispersion of targeted microbubbles is infused into the flow chamber at a known shear rate.

FIG. 2 provides a diagram of the modified flow chamber utilized in the deformability study. The target substrate (avidin) is coated onto a thin coverslip, which is placed into a standard 35 mm culture dish. A small view hole is cut through the culture dish to enable coupling of a high-magnification water immersion objective to the coverslip. The remainder of the flow chamber is assembled as described.

FIG. 3 provides a diagram of a wrinkle structure expected in certain embodiments of the invention. Pressurizing the microbubble causes the lipid monolayer (yellow) to buckle, forming a bilayered wrinkle. PEG molecules shown in red, immobilized targeting antibody shown in black.

FIGS. 4A and 4B show fluorescent (top panels) bright field (bottom panels) images of wrinkled and spherical microbubbles, respectively. Bubbles were statically adsorbed to plastic coverslip, and photographed at 100× with high-resolution digital camera. Epifluorescence allows visualization of the lipid shell, where the wrinkles occur, and bright field allows visualization of the gas core. Bar in lower right corner of top panels is 10 μm.

FIG. 5 shows microbubble size characteristics as described further in the Experimental: (A) Bar graph of median microbubble diameter of bubbles used in flow chamber experiments; (B) Coulter counter size distributions of spherical and wrinkled microbubbles; (C) Table of size parameters for wrinkled and spherical microbubbles.

FIG. 6 shows a graph illustrating site density of the target receptor P-selectin.Fc on a dish surface, as further described in the Experimental. Biotinylated Rb40.34 was incubated with dishes coated with appropriate quantity of P-selectin.Fc. Eu-conjugated streptavidin was to label the bound antibody, and the P-selecitn.Fc site density was quantified by time-resolved spectroscopy at λem=360 nm, λex=610 nm.

FIG. 7 shows the firm attachment efficiency of wrinkled and spherical microbubbles in flow chamber experiments, as described further in the Experimental. Firm attachment efficiency is defined as the percentage of microbubbles that remain attached for greater than 10 seconds relative to the total flux within binding distance. (A, B, C) Firm attachment efficiency of wrinkled and spherical microbubbles on variable P-selectin.Fc substrate densities; (D) Firm attachment efficiency for negative control conditions: Rb40.34 bubbles on casein, and Isotype control bubbles on 250 ng/dish P-selectin.Fc.

FIG. 8 shows the fraction of firmly attached microbubbles in flow chamber experiments, as described further in the Experimental. Firm fraction was calculated by dividing the firm attachment efficiency by the capture efficiency.

FIGS. 9A-9C show pause time durations for spherical and wrinkled microbubbles in flow chamber experiments, as described further in the Experimental. Frequencies were normalized against the total number of attachment events for each bubble type; exact values of normalized frequency are printed at the top of each column. Pause time data was compiled from the attachment efficiency flow chamber results (Experimental, Section 2.4.2) by calculating the duration of each attachment event off-line. Bubbles that adhered for >10 seconds were scored as ‘Firmly Attached’ and are indicated in column 10 seconds. The mean pause time for all transiently adherent bubbles was significantly below 10 seconds, thus justifying the use of 10 seconds as the cutoff between these two event populations.

FIG. 10 illustrates the deformability of wrinkled and spherical microbubbles under variable shear, as described further in the Experimental. Biotinylated, fluorescent wrinkled or spherical microbubbles were attached to a substrate of avidin at the indicated shear rate for 5 minutes. Images of adherent microbubbles were captured with a high-resolution camera at 100×. The deformation index was calculated as the degree of elongation in the direction of flow (x) divided by the length of the bubble perpendicular to flow (y). There was no deviation in pixel size between the x and y directions.

FIG. 11 shows P-selectin targeted microbubble accumulation in vivo for wrinkled and spherical microbubble populations. A mixed dispersion of 2.5×10⁶ each of wrinkled (Di-I labeled) and spherical (Di-O labeled) P-selectin-targeted or isotype control microbubbles was injected intravenously into c57B1/6 or P−/− mice, pre-treated with 500 ng murine TNF-alpha two hours prior to surgery. Microbubble adherence in the inflamed murine cremaster was determined by counting the number of wrinkled and spherical microbubbles adherent in venules in 10 optical fields. (A) Wrinkled and spherical microbubble adhesion in inflamed microcirculation; (B) physiological parameters.

### DETAILED DESCRIPTION

For the purposes of promoting an understanding of the principles of the invention, reference will now be made to the preferred embodiments illustrated in the following description and examples, and specific language will be used to describe the same. It will nevertheless be understood that no limitation of the scope of the invention is thereby intended, such alterations and further modifications in the preferred embodiments of the invention, and such further applications of the principles of the invention as described therein being contemplated as would normally occur to one skilled in the art to which the invention relates.

As disclosed above, the present invention provides microbubble compositions including microbubbles having membranes that incorporate modified surface features that may be useful, for example, in facilitating binding to a target surface or substance. The surface features may include non-spherical attributes such as crenations, folds, projections, or wrinkles, which can increase the deformability of the microbubble membrane. Such microbubble compositions can be incorporated into targeted ultrasound contrast agents and methodologies. Methods for preparing modified microbubble compositions include providing microbubbles having spherical membranes, and converting the spherical membranes to non-spherical membranes having surface features as mentioned above. Targeting substances can be incorporated into or otherwise attached to the membranes before or after their conversion from spherical to non-spherical forms.

Turning now to a discussion of membrane forming materials, a wide variety of such materials are known and can be used in preparing microbubble compositions of the invention. Illustratively, any compound or composition that aids in the formation and maintenance of the bubble membrane or shell by forming a layer at the interface between the gas and liquid phases may be used. The surfactant may comprise a single compound or any combination of compounds, such as in the case of co-surfactants. Preferred surfactants are lipids, including sterols, hydrocarbons, fatty acids and derivatives, amines, esters, sphingolipids, and thiol-lipids (each of which can solely constitute the microbubble shell or be used in a mixture with other lipids, phospholipids, surfactants, and detergents), nonionic surfactants, neutral or anionic surfactants, and combinations thereof.

Suitable surfactants include, for example, block copolymers of polyoxypropylene polyoxyethylene, sugar esters, fatty alcohols, aliphatic amine oxides, hyaluronic acid aliphatic esters, hyaluronic acid aliphatic ester salts, dodecyl poly (ethyleneoxy) ethanol, nonylphenoxy poly(ethyleneoxy)ethanol, hydroxy ethyl starch, hydroxy ethyl starch fatty acid esters, dextrans, dextran fatty acid esters, sorbitol, sorbitol fatty acid esters, gelatin, serum albumins, and combinations thereof.

Illustrative phospholipid-containing surfactant compositions include lecithins (i.e. phosphatidylcholines), for example natural lecithins such as egg yolk lecithin or soy bean lecithin, semisynthetic (e.g. partially or fully hydrogenated) lecithins and synthetic lecithins such as dimyristoylphosphatidylcholine, dipalmitoylphosphatidylcholine or distearoylphosphatidylcholine; phosphatidic acids; phosphatidylethanolamines; phosphatidylserines; phosphatidylglycerols; phosphatidylinositols; cardiolipins; sphingomyelins; fluorinated analogues of any of the foregoing; mixtures of any of the foregoing and mixtures with other lipids such as cholesterol. Phospholipids predominantly (e.g. at least 75%) comprising molecules individually bearing net overall charge, e.g. negative charge, may be used, for example as in naturally occurring (e.g. soy bean or egg yolk derived), semisynthetic (e.g. partially or fully hydrogenated) and synthetic phosphatidylserines, phosphatidylglycerols, phosphatidylinositols, phosphatidic acids and/or cardiolipins,

Illustrative nonionic surfactants include polyoxyethylene-polyoxypropylene copolymers. Example of such class of compounds are provided by the nonionic Pluronic surfactants. Polyoxyethylene fatty acids esters may also be used, including polyoxyethylene stearates, polyoxyethylene fatty alcohol ethers, polyoxyethylated sorbitan fatty acid esters, glycerol polyethylene glycol oxystearate, glycerol polyethylene glycol ricinoleate, ethoxylated soybean sterols, ethoxylated castor oils, and the hydrogenated derivatives thereof, and cholesterol.

Anionic surfactants, particularly fatty acids (or their salts) having 12 to 24 carbon atoms, e.g. oleic acid or its sodium salt, may also be used.

In addition to the surfactant(s), one may also incorporate other agents within the aqueous phase. Such agents may advantageously include conventional viscosity modifiers, buffers such as phosphate buffers or other conventional biocompatible buffers or pH adjusting agents such as acids or bases, osmotic agents (to provide isotonicity, hyperosmolarity, or hyposmolarity). Preferred solutions have a pH of about 7 and are isotonic. Microbubbles can be coated with a brush of a hydrophilic polymer, such as polyethylene glycol, polyvinylpyrrolidone, or polyglycerol, to assure low nonspecific attachment of the microbubbles to materials and surfaces.

As to gases that may be used in the core of the microbubbles, any biocompatible gas may be used (including mixtures). The gas may thus, for example, comprise air; nitrogen; oxygen; carbon dioxide; hydrogen; an inert gas such as helium, argon, xenon or krypton; a sulphur fluoride such as sulphur hexafluoride, disulphur decafluoride or trifluoromethylsulphur pentafluoride; selenium hexafluoride; an optionally halogenated silane such as methylsilane or dimethylsilane; a low molecular weight hydrocarbon (e.g. containing up to 7 carbon atoms), for example an alkane such as methane, ethane, a propane, a butane or a pentane, a cycloalkane such as cyclopropane, cyclobutane or cyclopentane, an alkene such as ethylene, propene, propadiene or a butene, or an alkyne such as acetylene or propyne; an ether such as dimethyl ether; a ketone; an ester; a halogenated low molecular weight hydrocarbon (e.g. containing up to 7 carbon atoms); or a mixture of any of the foregoing. At least some of the halogen atoms in halogenated gases can be fluorine atoms; thus biocompatible halogenated hydrocarbon gases may, for example, be selected from bromochlorodifluoromethane, chlorodifluoromethane, dichlorodifluoromethane, bromotrifluoromethane, chlorotrifluoromethane, chloropentafluoroethane, dichlorotetrafluoroethane, chlorotrifluoroethylene, fluoroethylene, ethylfluoride, 1,1-difluoroethane and perfluorocarbons, e.g. gases include methyl chloride, fluorinated (e.g. perfluorinated) ketones such as perfluoroacetone and fluorinated (e.g. perfluorinated) ethers such as perfluorodiethyl ether. The use of perfluorinated gases, for example sulphur hexafluoride and perfluorocarbons such as perfluoropropane, perfluorobutanes and perfluoropentanes, may be particularly advantageous in view of the recognized high stability in the bloodstream of microbubbles containing such gases.

As to the formation of the microbubble compositions, a variety of suitable methods are known. Sonication is preferred for the formation of microbubbles, i.e., through an ultrasound transmitting septum or by penetrating a septum with an ultrasound probe including an ultrasonically vibrating hypodermic needle. Optionally, larger volumes of microbubbles can be prepared by direct probe-type sonicator action on the aqueous medium in which microbubbles are formed in the presence of gas (or gas mixtures) or another high-speed mixing technique, such as blending or milling/mixing. Other techniques such as gas injection (e.g. venturi gas injection), mechanical formation such as through a mechanical high shear valve (or double syringe needle) and two syringes, or an aspirator assembly on a syringe, or simple shaking, may be used. Microbubbles can also be formed through the use of a liquid osmotic agent emulsion supersaturated with a modifier gas at elevated pressure introduced into in a surfactant solution.

When used to form the microbubbles, sonication can be accomplished in a number of ways. For example, a vial containing a surfactant solution and gas in the headspace of the vial can be sonicated through a thin membrane. The membrane can be made of materials such as rubber, Teflon, mylar, urethane, aluminized film, or any other sonically transparent synthetic or natural polymer film or film forming material. The sonication can be done by contacting or even depressing the membrane with an ultrasonic probe or with a focused ultrasound “beam.” The ultrasonic probe can be disposable. In either event, the probe can be placed against or inserted through the membrane and into the liquid. Once the sonication is accomplished, the microbubble solution can be withdrawn from the vial and delivered to the patient. Sonication can also be performed within a syringe with a low power ultrasonically vibrated aspirating assembly on the syringe, similar to an inkjet printer. Also, a syringe or vial may be placed in and sonicated within a low power ultrasonic bath that focuses its energy at a point within the container.

In certain embodiments of the invention, targeted microbubble compositions are provided. Such targeted microbubble compositions will include microbubbles having a ligand or targeting molecule included in or bound to the microbubble membrane, wherein the ligand or targeting molecule binds, optionally with specificity, to a target surface or material. Illustrative targeting molecules that may be used include, for example:

i) Antibodies and antibody fragments, including high specificity and high affinity antibodies. Conventional and/or genetically engineered antibodies may be employed. For human uses of microbubble compositions, human antibodies may be used to avoid or minimize possible immune reactions against the targeting molecule.

ii) Cell adhesion molecules, their receptors, cytokines, growth factors, peptide hormones, peptide mimetics, and pieces thereof.

iii) Non-peptide agonists/antagonists or non-bioactive binders of receptors for cell adhesion molecules, cytokines, growth factors and peptide hormones.

iv) oligonucleotides and modified oligonucleotides, including but not limited to, aptamers which bind DNA or RNA through Watson-Crick or other types of base-pairing.

v) Protease substrates/inhibitors. Proteases are involved in many pathological conditions.

vi) Various small molecules, including bioactive compounds known to bind to biological receptors of various kinds.

vii) Inactivated proteases as binding partners for their substrates.

Other peptide targeting molecules and lipopeptides thereof of particular interest for targeted ultrasound imaging include atherosclerotic plaque binding peptides; thrombus binding peptides, and platelet binding peptides.

Illustrative targeting molecule substances that may be targeted to particular types of targets and indicated areas of use for targetable diagnostic and/or therapeutic agents include antibodies to: CD34, ICAM-1, ICAM-2, ICAM-3, E-selectin, selectin, P-selectin, PECAM, CD18 Integrins, VLA-1, VLA-2, VLA-3, VLA4, VLA-5, VLA-6, GlyCAM, MAdCAM-1, fibrin, and myosin. These and other targeting molecule molecules are identified and discussed in U.S. Pat. No. 6,264,917, which is incorporated by reference herein generally and specifically for purposes of identifying such additional useful targeting molecule molecules.

Attachment of the targeting molecule to the membrane can be achieved in a number of ways. For example, the targeting molecules can be directly coupled via chemical crosslinking agents (e.g., via carbodiimide (EDC) or thiopropionate (SPDP)). Preferably, however, the targeting molecule is indirectly attached to the microbubble membrane through spacer arm molecules. Illustratively, the attachment structure may be defined as:
M-S-V
wherein M is the microbubble membrane, S is a spacer arm; and V is a targeting molecule.

Spacer arms (S) for use with the invention include a branched or linear synthetic polymer or a biopolymer like polyethyleneglycol (PEG), polyvinylpyrrolidone, polyoxyethylene, polyvinylpyridine, polyvinyl alcohol, polyglycerol, dextran, and starch. One end of the spacer arm molecule will be deposited at the gas-liquid interface, and the flexible polymer spacer arm will be extended in the liquid medium allowing improved interaction of the targeting molecule with the target.

The targeting molecule for use with the invention can be, for instance, any of those described above. A number of strategies may be employed for the attachment of targeting antibodies or other proteins to microbubbles through spacer arm molecules. For example, the spacer molecule can have one end that attaches to or incorporates within the microbubble membrane, and another end that contains a reactive or other binding group for direct or ultimate attachment to the targeting molecule. One illustrative strategy is based on the avidin-biotin bridge method. A spacer molecule, for instance PEG, has biotin attached to one end and a microbubble-membrane forming molecule (e.g. a phospholipid) attached to the other end to form a membrane anchor. The membrane anchor is incorporated into the microbubble membrane, leaving the spacer arm-biotin portion extending into the liquid phase. Avidin is then bound to the spacer arm-biotin sites, and free binding sites on avidin are then used to bind biotinylated antibodies or other targeting molecules to the spacer arms. Use of such avidin-biotin combinations can provide universal reagents, to which any biotinylated antibody of interest can be later attached for a specific, targeted application. Alternatively, a metal-chelating molecule can be attached to the microbubble surface, so that attachment of His-tagged recombinant proteins can be readily achieved using known, standard coupling schemes. These and other methods for attachment of targeting molecules to microbubble membranes through spacer arm molecules are known and can be used in the present invention. Additional such information may be found, for instance, in U.S. Pat. No. 6,245,318, which is incorporated herein by reference.

Microbubbles in accordance with the invention can be rendered non-spherical in a number of ways. Generally, a spherical microbubble can be modified to a non-spherical microbubble by reducing the volume of entrapped gas while at the same time retaining the same or substantially the same amount of membrane material in the microbubble. This results in an excess of membrane material over that needed to encapsulate the gaseous core, and this excess membrane material provides an increased deformability to the microbubble relative to the starting, spherical microbubble (which will be of larger size), and also relative to a spherical microbubble of the same size. Upon attachment to a target substrate, this excess membrane material can allow a larger microbubble: substrate contact area to form with respect to that of a spherical microbubble. The excess material will typically form protrusions from the microbubble membrane, which can be described as crenations, folds, wrinkles, or other irregularities. When preparing non-spherical microbubbles from spherical microbubbles, the hydrostatic pressure or other method for removing gas from within the microbubbles is desirably applied so as to remove at least about 10% of the gas from within spherical microbubbles converted to non-spherical microbubbles, more preferably at least about 20% of the gas. Typically, such methods will be used to remove in the range of about 10% to 80% of the core gas from the spherical microbubbles, more typically in the range of about 20% to 70%. Moreover, such methods can be applied to as to achieve these levels of gas core reduction in at least about 20% of the microbubbles in an original spherical microbubble population, preferably greater than about 50%, and more preferably in the range of about 80% to about 100%. If desired, a mixed spherical/non-spherical microbubble population resultant of such methods may be treated to separate spherical from non-spherical microbubbles and achieve a more enriched, non-spherical microbubble population.

Illustratively, non-spherical microbubbles can be prepared by subjecting spherical microbubbles to pressure, e.g. to hydrostatic pressure in a closed container. Thus, microbubbles optionally containing a targeting molecule attached to the surface directly or indirectly, can be dispersed in an aqueous phase and placed in a container (e.g. syringe) with a 3-way stopcock valve attached. The stopcock valve is closed, the syringe plunger is partly pressed to create the increased pressure inside the unit (1-20 psi). This pressure is uniformly transferred throughout the volume, thus creating overpressurization inside the microbubbles, and reduction in gaseous volume occurs. The excess of the microbubble shell is protruded out of the plane of gas-liquid interface into the aqueous phase, creating crenation irregularities in the form of folds/flaps. The material is incubated at increased pressure for about 5 seconds to 100 minutes, during which time microbubble gas under pressure is partially diffusing out of the microbubble and dissolving in the surrounding medium, making the reduction in volume of the microbubble ensemble permanent after the plunger is released and the microbubble is brought back to atmospheric pressure conditions.

Crenated microbubbles can also be prepared by first preparing spherical microbubbles from a mixture of gases including a water soluble gas and a water insoluble gas, including the gradation of solubility, e.g. decafluorobutane and air. An aqueous medium into which the microbubbles are to be dispersed can first be degassed, e.g. by vacuuming it, or preferably by sparging with an insoluble gas to remove soluble gas from the medium. As a result, when the microbubbles are dispersed into the degassed medium, the encapsulated microbubble volume is reduced as soluble gas is driven from the inside of the bubble to the medium, and membrane surface crenations are generated. Alternatively, insoluble gas can be sparged directly through a dispersion of microbubbles prepared from a soluble-insoluble gas mixture, resulting in soluble gas loss from within the bubbles, volume reduction, and the creation of crenations. Optionally, the crenation process may be designed to take place after the microbubble agent is administered into the bloodstream of the patient, for example by exposing the microbubbles to ultrasonic radiation.

Microbubble compositions of the invention will desirably be prepared to have a substantial percentage (i.e. about 20% or more) of non-spherical microbubbles. More preferably, microbubble compositions of the invention will contain predominantly (i.e. greater than 50%) non-spherical microbubbles, and even more preferably about 80% to essentially 100% non-spherical microbubbles. Such enrichment in non-spherical microbubbles can occur as a result of the techniques used to originally prepare a microbubble composition, and/or through purification techniques subsequently applied. Further, the microbubble compositions of the invention can have substantially uniform size distributions for the microbubbles. For example, the compositions can be prepared and/or purified such that at least about 30% of the non-spherical microbubbles have a diameter within about 3 micrometers (microns) of the mean diameter of the population, more preferably at least about 50%. In addition or alternatively, about 30% of the non-spherical microbubbles in the composition can have a diameter within the range of about 1 to about 7 micrometers, more preferably at least about 50%.

It will be understood that the targeting molecule (e.g. antibody) can be attached to the microbubbles at any suitable time, including before, during or after the procedure used to render the microbubbles non-spherical.

Microbubbles having both spacer-arm-attached targeting molecules and deformable, non-spherical (e.g. crenated, wrinkled, folded, etc.) membranes present particular, advantages when targeting binding to a surface. The availability of the targeting molecule to bind to the target is enhanced both by the deformability of the membrane and the flexibility of the spacer arm to accommodate conformation of the receptor at the binding site or sites.

The microbubble compositions of the invention can be formulated into pharmaceutical compositions such as diagnostic or therapeutic compositions for enteral or parenteral administration, and can also be used for in vitro imaging, delivery or separation processes, among others. For use in ultrasound imaging, these compositions can contain an effective amount of the ultrasound agent along with conventional pharmaceutical carriers and excipients appropriate for the type of administration contemplated. Parenteral compositions may be injected directly or mixed with a large volume parenteral composition for systemic administration. Such solutions also may contain pharmaceutically acceptable buffers and, optionally, electrolytes such as sodium chloride.

Diagnostic compositions of the invention will be administered in doses effective to achieve the desired enhancement of the ultrasound image. Such doses may vary widely, depending upon the particular agent employed, the organs or tissues which are the subject of the imaging procedure, the imaging procedure, the imaging equipment being used, and the like. The diagnostic compositions of the invention can be used in the conventional manner. The compositions may be administered to a patient, typically a warm-blooded animal, either systemically or locally to the organ or tissue to be imaged, and the patient then subjected to the imaging procedure. Illustratively, ultrasonic imaging of any organ in which the receptor for the targeting molecule is present on the vascular surface can be undertaken in accordance with the invention.

Therapeutic compositions of the invention will include at least one active agent, such as a therapeutic pharmaceutical agent, e.g. a drug substance, to be delivered to the patient. Such compositions can be delivered by any suitable route, including enteral or parenteral administration, local or systemic. Illustrative drug substances useful for these purposes include for example those disclosed in U.S. Pat. No. 6,264,917, which is incorporated by reference herein generally and specifically for purposes of identifying such useful drug substances.

Kits can be prepared for use in making the microbubble preparations of the present invention. These kits can include a container enclosing the gas or gases described above for forming the microbubbles, the liquid, and the membrane-forming surfactant. The container can optionally include adaptations for exerting hydrostatic pressure upon a prepared, spherical microbubble composition, e.g. as in a syringe. Alternatively, the container can contain the gas or gases, and the surfactant and liquid can be added to form the microbubbles. Still further, the surfactant can be present with the other materials in the container, and only the liquid needs to be added in order to form the microbubbles. Where a material necessary for forming the microbubbles is not already present in the container, it can be packaged with the other components of the kit, preferably in a form or container adapted to facilitate ready combination with the other components of the kit.

Microbubble compositions of the invention can also be used as affinity isolation reagents, for example based upon the microbubble upward mobility in an aqueous or other liquid medium in normal or artificial gravity fields. When so used, the compositions can provide enrichment of a target substance or entity (e.g. cells, microparticles), for example from an environmental sample or biological fluid or other sample.

To promote a further understanding of the invention and its features and advantages, the following Experimental is provided. It will be understood that this Experimental is illustrative, and not limiting, in nature.

### EXPERIMENTAL

Section 1: Materials and Methods

1.1 Reagents

The P-selectin Fc fusion protein (P-selectin.Fc) was obtained in lyophilized form from R&D Systems (Minneapolis, Minn.). Aliquots of P-selectin.Fc were made by dissolving 750 μg of the lyophilized protein in 15 μL of Dulbecco's Phosphate Buffered Solution (DPBS) (Invitrogen, UK), which were frozen in liquid nitrogen and stored at −20° C. for less than 4 months. Prior to biotinylation, the anti-P-selectin antibody Rb40.34 stock was dialyzed in DPBS overnight using a 10,000 Dalton MWCO dialysis cartridge (Pierce, Rockford, Ill.). N-hydrosuccinimido-biotin (NHS-biotin) and streptavidin for the antibody-microbubble linkage system were obtained from Sigma (St. Louis, Mo.). Eu-labeled streptavidin and DELFIA solution were obtained from Wallac Oy (Turku, Finland). Advanced Protein Assay Reagent used in determining antibody concentrations was obtained from Cytoskeleton, Inc (Denver, Colo.). Blocker Casein in TBS used to block non-specific adhesion in flow chamber experiments was obtained from Pierce (Rockford, Ill.). Tween-20 was obtained from J.T. Baker (Phillipsburg, N.J.). Isoton-II used as a diluent for the Coulter counter was obtained from Beckman Coulter (Miami, Fla.). Kimura [0.05% wt/vol toluidine blue, 0.9% NaCl in 22% ethanol, 0.03% light-green SF yellowish, saturated saponin in 50% ethanol, and 0.07 M phosphate buffer, pH 6.4; all from Sigma (St. Louis, Mo.)] was used to stain leukocytes for blood counts (Olsen and et al, 2001).

1.2 Inverted Parallel Plate Flow Chamber

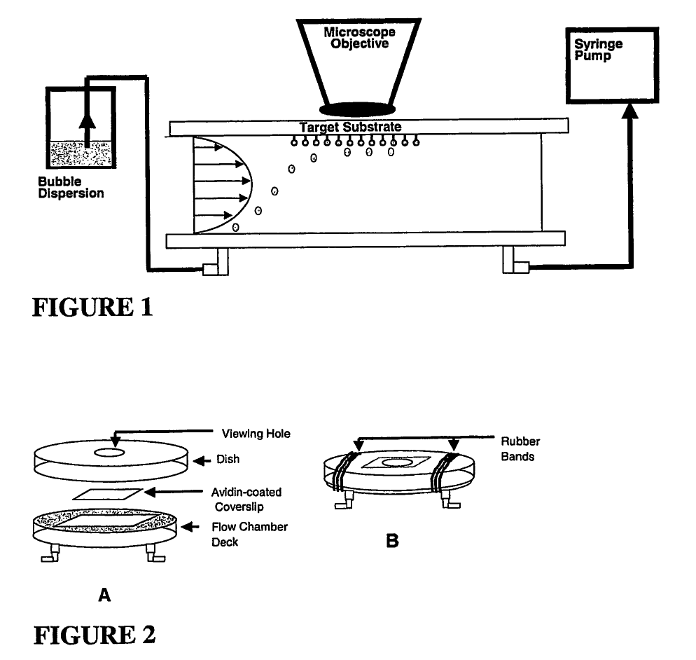

In vitro adhesion efficiency experiments were performed with a parallel plate flow chamber (GlycoTech, Rockville, Md.). The distance between the top and bottom plates, determined by the gasket thickness, was 0.254 mm, and the flow path width was 2.5 cm (gasket “B”). 35 mm diameter polystyrene dishes (Corning, Corning, N.Y.), on which the P-selectin.Fc substrate was plated, served as the top plate of the flow chamber. Because of the buoyancy of the microbubbles, the flow chamber was used inverted by means of a specially fabricated holder. The dish was secured onto the flow chamber by vacuum suction and circumferential rubber bands, and the entire flow chamber was inserted into the inverted holder. A syringe pump (Harvard Apparatus, Cambridge, Mass.) in the aspiration mode was used to draw the microbubble dispersion through the flow chamber at defined shear rates. Experiments were visualized with a Leitz Laborlux II microscope (Rockleigh, N.J.) using partial epifluorescence with a 40× objective (Olympus, Tokyo, Japan). Data from the flow chamber experiments was recorded on standard VHS cassettes (Sony, Tokyo, Japan) or digital video cassettes (Sony, Tokyo, Japan) for subsequent off-line analysis. A diagram of the inverted parallel plate flow chamber is presented in FIG. 1.

1.3 Mice

Five male C57B1/6 and one P-selectin knockout (P−/−) mice were obtained from Hilltop Labs (Scottsdale, Pa.) and housed in the U.Va small animal facility. All mice were between the ages of 8 and 12 weeks, and appeared healthy. All animal experiments were approved by the University of Virginia institutional animal care and use committee (Protocol #2474).

1.4 Biotinylation of Antibody

Rb4O.34 obtained from hybridoma supernatant was dialyzed overnight in DPBS at 4° C. 45 μg NHS biotin was dissolved in 10 μL DMSO for each 2 mg of monoclonal antibody (mAb) to be biotinylated. The mixture was allowed to incubate 2 hrs at 4° C., followed by dialysis overnight to remove unbound biotin.

1.5 P-Selectin Adsorption

35 mm polystyrene dishes were washed with methanol and allowed to air dry prior to attachment of the P-selectin.Fc protein. A 1 cm diameter circle was drawn with a felt pen in the center of each washed plate, using the same circular template for all dishes. The required mass (25, 150, or 250 ng) of the P-selectin.Fc fusion protein was diluted to 200 μL in DPBS and adsorbed to the circled area of the dish. The dishes were then covered with the provided lid and a wet paper towel to prevent excess evaporation. Binding occurred overnight at 4° C. Dishes were subsequently washed five times with 0.05% Tween-20 in DPBS to remove unbound protein. To prevent non-specific adhesion to the dish surface, dishes were blocked by incubation with 2 mL of casein in TBS for at least 2 hrs at room temperature. All dishes were blocked and used on the same day, and defective dishes that did not seal properly in the flow chamber were discarded. As a negative control, some blocked dishes were incubated with 1.0 mg Rb40.34 in 1.0 mL DPBS for at least 30 minutes and washed 5 times with 0.05% Tween-20. Alternatively, some dishes were incubated with casein instead of P-selectin.Fc and subsequently washed and blocked.

1.6 Microbubble Preparation

Microbubbles used in these experiments were composed of a decafluorbutane (C₄F₁₀) gas core encapsulated by a lipid shell of distearoylphosphatidylcholine (DSPC). A brush of (poly)ethyleneglycol (PEG) surrounded the lipid shell. The biotinylated C₄F₁₀-filled microbubbles were prepared as described previously (Kim et al, 2000; Lindner et al, 2001). Briefly, C₄F₁₀ gas was dispersed into an aqueous dispersion of phosphatidyl choline, PEG stearate and biotin-PEG-DSPC by high-output sonication. This formed lipid-encapsulated C₄F₁₀ microbubbles; lipids not incorporated into microbubbles were removed by repeated centrifugal flotation. Two populations of microbubbles were prepared: fluorescently labeled, in which a fluorescent probe (Di-O or Di-I) was incorporated into the lipid shell, or unlabeled. Each microbubble population was stored separately at 4° C. for up to 2 months, with minimal gas loss as detected by microscopic observation and Coulter size analysis.

Approximately 250×10⁶ bubbles of each population (Di-I labeled and unlabeled) were washed five times in a bucket centrifuge at 500 RPM for 5 minutes and re-suspended in 2.0 mL C₄F₁₀-saturated DPBS. Washes were performed in 5.0 mL syringes from which the plungers had been removed. Centrifugation forced the bubbles to cake at the top of the syringe (as described in U.S. Pat. No. 6,245,318), and the infranatant was removed from the bottom of the syringe by means of a Luer-lock valve.

After two washes to remove small microbubbles and free and micellar lipids, the Di-I labeled microbubble population was partially crushed as follows. The 2.0 mL dispersion was drawn into a 10 mL syringe containing 8.0 mL of air. The syringe was closed and depressed such that the volume was decreased to 6.5 mL and 750 mmHg was distributed uniformly among all bubbles in the dispersion. Pressure applied to the dispersion was measured with an electronic manometer (Dwyer, Michigan City, Ind.). Repeated washings eliminated any empty shell fragments from the wrinkled population, and produced a more uniform size distribution for both populations, as measured by a Coulter counter (Beckman-Coulter, Miami, Fla.).

3 μg of streptavidin per 10⁷ microbubbles were added to each microbubble population. Following 30 minutes of incubation on ice, the microbubbles were washed twice to remove unbound streptavidin. 7.5 μg of biotinylated Rb40.34 antibody per 10⁷ microbubbles were added to each population. The microbubbles were incubated for 30 minutes on ice and washed three times to remove unbound antibody. This protocol typically yielded between 20 and 50×10⁶ antibody-coupled microbubbles in 2.0 mL of each population. Population characteristics, including concentration and mean diameter, were obtained for both spherical and wrinkled microbubbles after each wash.

1.7 P-Selectin.Fc Site Density Determination

Known concentrations (25, 150, or 250 ng in 200 μL) of P-selectin.Fc were adsorbed to 35 mm dishes as described above. Following 2 hrs of blocking in casein, 0.96 ug of biotinylated Rb40.34 in 1.0 mL DPBS was added to each dish. The amount of solvent was sufficient to cover the entire surface of the dish. After 30 minutes of incubation at room temperature, unbound antibody was removed by 7 washes with 0.05% Tween-20, and 0.1 μg Eu-labeled streptavidin in 1.0 mL was added to each dish. After 30 minutes of incubation at room temperature, the dish was washed 7 times to remove unbound streptavidin. 0.9 mL of DELFIA enhancement solution was added to each plate and incubated for 5 minutes at room temperature. The reactant was collected from each dish and placed in a 96-well microtitre plate (300 μL per well) for time-resolved spectrofluoroscopy using a SPECTRAmax Gemini XS dual-scanning microplate spectrofluorometer (Molecular Devices, Sunnyvale, Calif.). Plates were excited at 360 nm and read at 610 nm during a 250-1250 μs timer interval. Scanning plates incubated with casein alone determined the level of non-specific antibody adhesion. Rb40.34 biotinylation was determined to be approximately 0.3 mole biotin per mole antibody using an avidin-HABA displacement method (Green, 1965). Site densities of adsorbed P-selectin.Fc were calculated assuming 1-to-1 binding of Rb40.34 to each head of the P-selectin.Fc dimer, and 1-to-1 binding of streptavidin to Rb40.34.

1.8 Flow Chamber Protocol

An equal number of antibody-labeled wrinkled and spherical microbubbles were mixed and diluted to approximately 3×10⁶ bubbles/mL in C₄F₁₀-saturated DPBS buffer. Saturation of the microbubble buffer prevented excess gas movement into or out of the microbubbles. The mixed microbubble dispersion was continuously stirred with a magnetic stir bar throughout each experiment to ensure homogeneity. The mixed microbubble dispersion was drawn into the flow chamber at the indicated shear rate through 10 cm of 0.6 mm-diameter tubing. A single field of view (110 μm×150 μm) close to the center of the 1 cm diameter circle in which the P-selectin.Fc had been adsorbed was observed for the duration of each experiment. Epifluorescent illumination was maintained with low-intensity transillumination, which enabled discrimination between fluorescently-labeled wrinkled and unlabeled spherical microbubbles. Each dish was observed at a single shear rate for the duration of the experiment. Each experiment lasted approximately 10 minutes, or until the field of view became saturated with bound microbubbles. A total flux of between 75 to 300 microbubbles of each population was observed over a single field of view for each dish.

1.8.1 Attachment Efficiency

Attachment efficiency was determined off-line by counting the number of adherent microbubbles for each population. Microbubbles were classified as either transiently adherent, in which case the bound microbubble detached within 10 seconds, or firmly adherent, in which case the bound microbubble persisted for greater than 10 seconds. The number of adherent microbubbles was normalized by the flux of microbubbles within binding distance (1 microbubble diameter) of the surface to yield attachment efficiency.

1.8.2 Microbubble Pause Time

Distributions of the duration of microbubble adhesive events were compiled for the flow chamber experiments described above. The video was replayed at ⅓ speed to enable detection of short lived attachment events. An event duration was calculated by counting the number of frames in which the bubble remained stationary and dividing by the video frame rate. Microbubbles that remained adherent for at least 10 seconds were scored as firmly attached, and were not analyzed beyond 10 seconds (although the majority of these bubbles were observed to remain bound for the remainder of the experiment). The pause times of transiently bound bubbles were pooled at each P-selectin.Fc site density and plotted.

1.9 Intravital Microscopy

Mice were given 0.5 μg murine tumor necrosis factor (TNF-α) (Sigma, St. Louis, Mo.) intrascrotally 2 hours before surgery to induce optimal expression of P-selectin in the cremaster muscle. Mice were anesthetized with an intraperitoneal injection of 125 mg/kg body weight ketamine (Parke-Davis, Morris Plains, N.J.), 12.5 mg/kg body weight xylazine (Phoenix Scientific, St. Joseph, Mo.), and 0.025 mg/kg body weight atropin sulfate (Elkins-Sinn, Cherry Hill, N.J.). Body temperature was maintained at 38° C. during preparation with an electric heat pad. The trachea was intubated with PE 90 tubing (Becton Dickinson, Sparks, Md.) to promote spontaneous respiration, and the right jugular vein was cannulated using PE 20 tubing. The microbubble dispersion was administered through the jugular cannula, and blood samples taken at the end of each experiment were taken from the carotid artery.

One cremaster muscle was exteriorized and continuously superfused with an isothermic bicarbonate-buffered solution (131.9 mM NaCl, 18 mM NaHCO₃, 4.7 mM KCL, 2.0 mM CaCl₂*2H₂O), 1.2 mM MgCl₂) equilibrated with 5% CO₂. The exteriorized cremaster was pinned to the microscope stage (Carl Zeiss, Thornwood, N.Y.) and placed beneath a saline immersion objective (SW 40/0.8 num aperture) for intravital microscopy. The preparation was visualized with a high-resolution CCD camera (Dage-MI, Inc, Michigan City, Ind.) and recorded on VHS videocassette for off-line analysis. The cremaster vasculature was epifluorescently illuminated with filters for either Di-I labeled microbubbles (λₑₓ=480 nm and λₑₘ=535 nm) or for Di-O labeled microbubbles (λₑₓ=545 nm and λₑₘ=610 nm) to reveal the presence of adherent microbubbles, and subsequently transilluminated to locate the adherent microbubbles with respect to the vascular space.

2.5×10⁶ microbubbles of each population (Di-I labeled wrinkled and Di-O labeled spherical) were injected through the jugular cannula. The number of adherent microbubbles of each type was determined for 10 optical fields between 4 and 20 minutes after injection. The centerline velocity (V_{c}) of each vessel was determined using a dual-slit photodiode (CircuSoft Instrumentation, Hockessin, Del.). The wall shear rate (s⁻¹)was determined by Eq. 15:

$$γ = 10.6 \cdot ( \frac{{V}_{c}}{d} ) \qquad Eq . 1$$

where γ is wall shear rate (s⁻¹), V_{c} is centerline velocity (μm/s), and d is the diameter of the vein (μm) (Reneman et al, 1992). Only venules between 15 and 55 μm were analyzed. Blood samples were taken in a 10 μL capillary tube at the end of each experiment. The blood sample was mixed with 90 μL Kimura to stain leukocytes, and counted in an hemocytometer.
1.10 Microbubble Deformation

Biotinylated microbubbles were prepared as described in the Experimental above, using Di-I labeled shell for both populations. A modified flow chamber was constructed, which enabled transillumination with a 100× water-immersion objective. A circular hole 1 cm in radius was cut out of a standard Corning 35 mm polystyrene dish and a plastic coverslip, on which 200 μg of avidin had been adsorbed, was fitted over the hole. The modified dish was held onto the flow chamber deck by several circumferential rubber bands and the usual flow chamber vacuum. A diagram of the modified flow chamber is presented in FIG. 2. Each microbubble population was infused into the flow chamber separately. Following 5 minutes of infusion at 45 s⁻¹, during which time sufficient bubbles for analysis had adhered, the bubble dispersion was replaced with C₄F₁₀-saturated DPBS. Approximately 5 fields of view encompassing 5-20 adherent microbubbles were examined and photographed. The shear rate was then increased to 122.5 s⁻¹ for 5 minutes, at which time 5 more fields of view were examined. The shear rate was increased to 306.3 s₋₁ for five minutes, and five fields of view were again obtained. Care was taken to align the camera in the direction of flow. The deformation index of each adherent microbubble was calculated as the length of the microbubble along its longest axis in the direction of flow divided by its length along its largest axis perpendicular to flow.

1.11 Echogenic Response

Crenated microbubbles were prepared by hydrostatic pressurization as described above. A dispersion of 24.0×10⁶ or 120×10⁶ spherical (untreated) or wrinkled microbubbles was infused into a 0.5 L bag of injection-grade 0.9% NaCl. Echogenicity of the microbubbles in the saline bag was determined with a linear ultrasound probe using harmonic imaging.

In another experiment, avidin (Sigma) was plated onto 35 mm dishes at various concentrations. Approximately 5×10⁶ crenated or round (untreated) microbubbles coated with biotin were infused over the avidin-coated dishes in the parallel plate flow chamber at a low shear rate. The number of bubbles bound to each dish was ascertained visually. The bound microbubbles were prevented from contacting air by disassembling the dish from the flow chamber in a bath of C₄F₁₀-saturated PBS following each experiment. Each dish was subsequently imaged with a linear ultrasound probe placed in the PBS bath. Echogenicity of the bubbles bound to each dish was again determined visually on the screen of an Agilent Sonos5500 medical ultrasound imaging system.

1.12 Statistical Analysis

The attachment efficiency of spherical and wrinkled microbubbles was compared at each of the three applied wall shear rates for the flow chamber experiments. A paired t-test was performed using the Excel v 9.0 spreadsheet package (Microsoft). Significance was tested at p=0.05. The mean number of adherent microbubbles in the 10 examined venules was compared in the in vivo experiments. The same paired-sample t-test was performed for these data.

1.13 Targeted Extraction of Microparticles or Cells by Wrinkled Ligand-Microbubbles.

1 ml of washed biotinylated crenated microbubbles prepared as described in Example 1-5 can be mixed with ˜1.108 fluorescent polystyrene latex beads that are coated with streptavidin (1 um diameter, Molecular Probes, Eugene, Oreg.), and incubated with mixing for 30 min. Material can be then placed in a vertically positioned syringe or flask and incubated at normal gravity or centrifuged in a bucket rotor at ˜500 rpm (r=15 cm) to achieve flotation of microbubbles and attached microparticles. Alternatively, microbubbles can be coated with an antibody or another ligand or targeting molecule and mixed with the mixture of cells some of which carry the specific antigen. Selective binding of antigen-carrying cells to microbubbles will result in the flotation of bubble-cell complexes and selective enrichment of the supranatant microbubble layer with the cells of interest and devoid of cells without the antigen of interest, that would not bind to the bubbles and sediment in the aqueous medium. The presence of crenations on the microbubble surface facilitates the attachment of microbubbles to target cells and improves binding efficacy and attachment force.

Section 2: Results

2.1 Microbubble Crushing

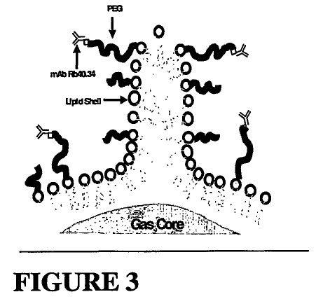

The lipid shell presents a barrier to gas diffusion across the bubble surface. Slowly and uniformly applying a positive pressure across the microbubble forces diffusion of its gaseous content across the lipid shell. The surface area of the lipid monolayer shell is now in excess of the surface of the gas core, and the shell buckles outward to form wrinkles and folds. These wrinkles form outward-projected bilayers. If the wrinkled microbubbles are stored in a solution saturated with C₄F₁₀, the concentration gradient opposes re-inflation; thus, the wrinkles are stable. A diagram of the expected wrinkle structure is presented in FIG. 3. Pressurizing the microbubble causes the lipid monolayer (yellow) to buckle, forming a bilayered wrinkle. PEG molecules shown in red, immobilized Rb40.34 antibody shown in black.

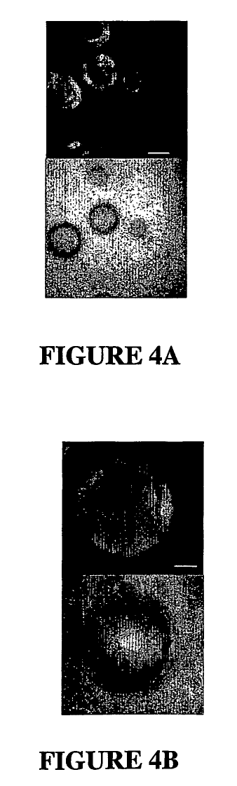

Wrinkles were generated on the surface of microbubbles by the application of a positive hydrostatic pressure. The existence of these wrinkles was confirmed by fluorescence microscopy, which revealed defined ridges and folds similar to the microfolds found on the surface of leukocytes. Wrinkles were observed to extend up to 0.3 μm. Incorporation of the fluorescent probe di-I allowed visualization of the lipid shell under fluorescent epi-illumination (FIGS. 4A and 4B, top panels), while bright-field transillumination showed only the gas core (FIGS. 4A and 4B, bottom panels). The bright-field images provided confirmation that the structures seen under fluorescence were in fact bubbles, and not simply empty lipid shells. In contrast to the wrinkled microbubbles, the spherical microbubbles appeared round under fluorescence. These wrinkles were stable for several hours when the microbubbles were stored in C₄F₁₀-saturated medium.

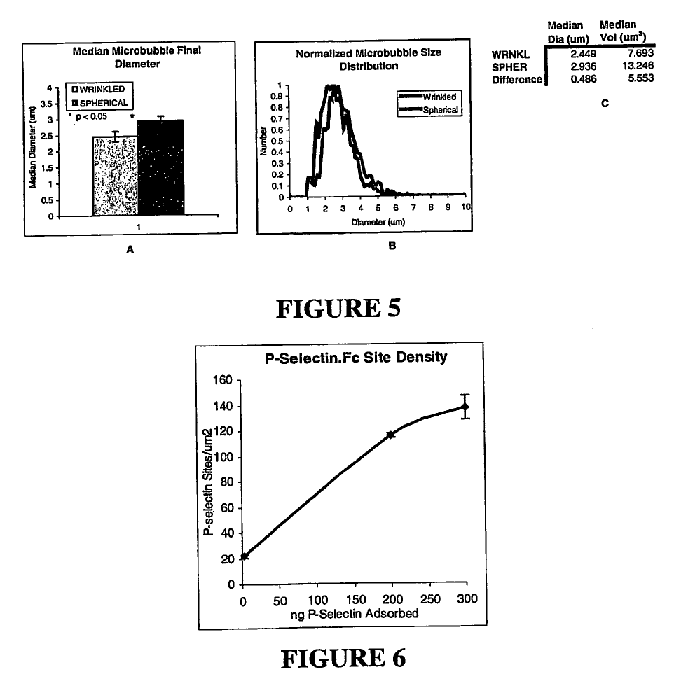

The Coulter multisizer was used to obtain size characteristics of the two microbubble populations. The Coulter counter calculates the size of a particle by measuring its electrical conductivity. Thus, the size distributions obtained from the Coulter counter report the size of the gas core of each microbubble population, and do not account for the irregularities in the surface of the wrinkled bubbles. A difference in diameter of approximately 0.5 μm between the two microbubble populations was apparent from the measured size distributions FIG. 5). This size difference confirms that the wrinkled microbubbles have lost some of their gaseous volume, and thus converted some lipid surface area to flat, volumeless folds. The gaseous volume of a microbubble of either population may be calculated as the volume of a sphere of diameter equal to that reported by the Coulter counter. The volume of gas in an average wrinkled microbubble was 7.7 μm₃, while that of an average spherical microbubble was 13.2 μm³. Hence, 5.6 μm₃ of C₄F₁₀ was excluded from an average wrinkled microbubble, which represents a volume loss of 41%.

2.2 Site Density of P-Selectin.Fc on Dish Surface

The flow chamber experiments were performed on a substrate of recombinant murine P-selectin (P-selectin.Fc) at three different concentrations. A graph of site density versus mass P-selectin adsorbed was created for the P-selectin.Fc protein on the dish surface, and is presented in FIG. 6. Site densities for 25 ng, 150 ng, and 250 ng were, respectively, 31, 97, and 133 sites/μm². The graph was non-linear, and appeared to saturate at approximately 300 ng P-selectin per 0.785 cm² (area of 1 cm circle). The non-specific background adhesion produced a signal equivalent to 14 sites/μm².

2.3 Attachment Efficiency In Vitro

2.3.1 Firm and Transient Attachment Efficiency

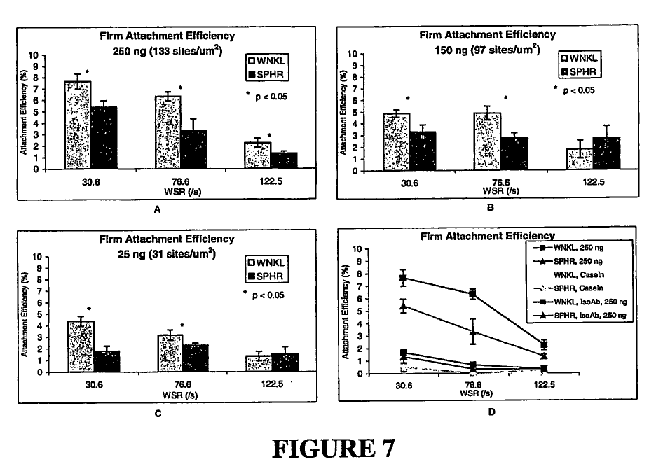

Two types of adhesive events were examined in this project: transient attachments, and firm attachments. Firm attachments are favorable for imaging because they allow the accumulation of microbubbles in the intended target area. The attachment efficiency of P-selectin targeted microbubbles was examined in the flow chamber at three shear rates for three P-selectin.Fc site densities. The results are shown in FIG. 7. Firm attachment efficiency was found to decrease approximately linearly with shear rate, from a maximum of 7.6% at the highest P-selectin concentration (250 ng/plate) at 33.6 s⁻¹ to 2% at the lowest concentration (25 ng/plate) at 122 s⁻¹ (FIG. 7A, B, C). At each of the site densities examined, the wrinkled microbubbles exhibited significantly (p<0.05) greater firm attachment efficiency than spherical microbubbles at 33.6 s⁻¹ and 76.6 s⁻¹. There was no significant difference between wrinkled and spherical microbubble firm attachment efficiency at 122 s⁻¹ on concentrations of 150 ng and 25 ng per dish. Targeting wrinkled and spherical microbubbles with an isotype control antibody (Iso Ab), or infusing P-selectin-targeted microbubbles over a substrate of pure casein resulted in negligible firm attachment (<2%) (FIG. 7D).

Transient attachment efficiency decreased with shear rate for both spherical and wrinkled microbubbles. Transient attachment efficiency was significantly higher for spherical microbubbles except under a shear of 122.5/s, where there was no statistical difference shown in this work.

2.3.2 Fraction of Firm Attachment Events

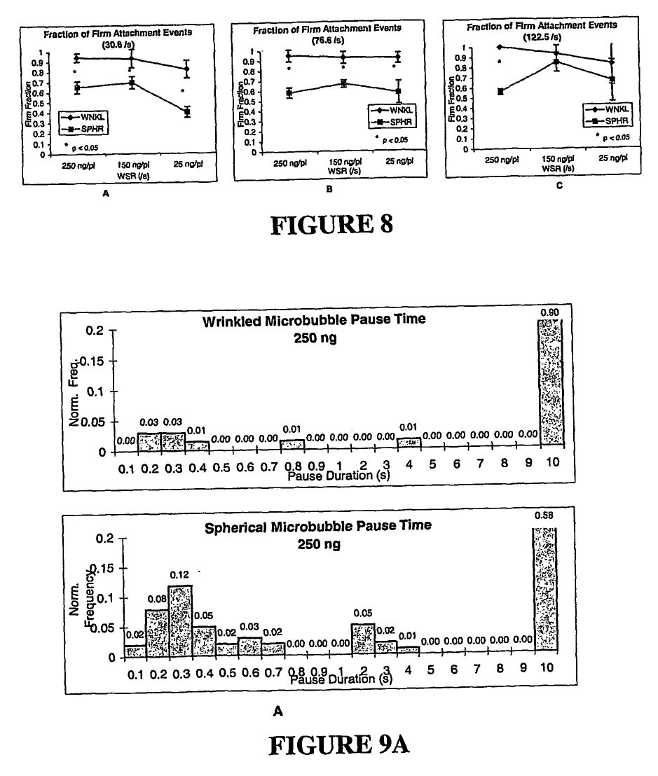

The fraction of firm attachment events may be calculated by dividing the firm attachment efficiency by the capture efficiency. This fraction represents the tendency of microbubbles to form firm (as opposed to transient) attachment events, or equivalently, how well capture events are stabilized and converted to firm adhesion (FIG. 8). The firm attachment fraction was plotted as a function of P-selectin.Fc site density at each shear rate. The firm attachment fraction of wrinkled microbubbles was relatively constant on 250 and 150 ng P-selectin at each of the shear rates, and decreased slightly on 25 ng P-selectin. The spherical microbubbles showed a similar site-dependent trend, although under all conditions the fraction of firm attachment was significantly lower than that of the wrinkled bubbles.

2.3.3 Pause Time Distribution

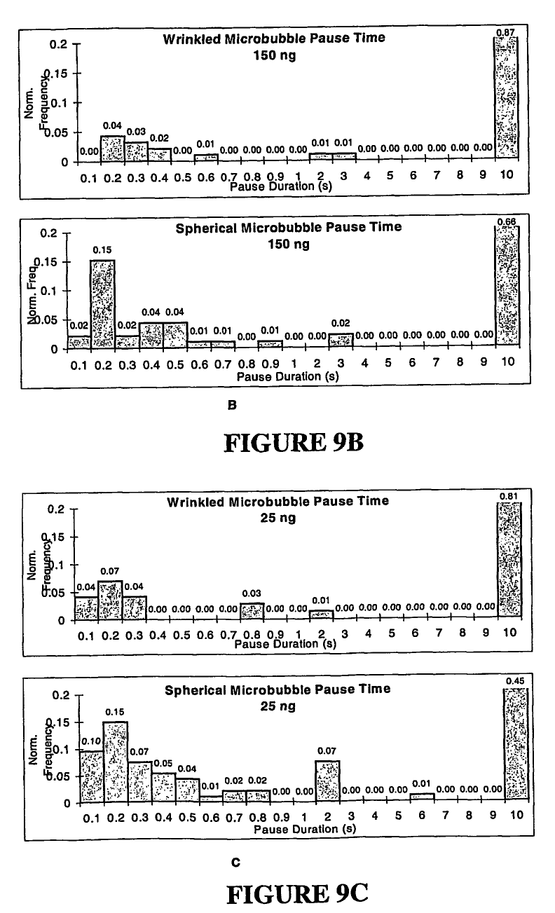

Two types of attachment events were examined here: firm attachment events, in which the bubble adhered for at least 10 seconds, and transient attachments, in which the bubble adhered for less than 10 seconds before becoming dislodged. 10 seconds was chosen as the cutoff for firm attachment, although the majority of bubbles that adhered for at least 10 s were observed to remain bound for much longer, usually the entire duration of the flow chamber experiment. Analysis of the pause-time distributions from these flow chamber experiments revealed that transient and firm attachment events form two distinct populations, and occur in different proportions for wrinkled and spherical bubbles (FIGS. 9A-9C). These results justify the use of 10 seconds as the cutoff time between firm and transient adhesion.

2.4 Deformation Index

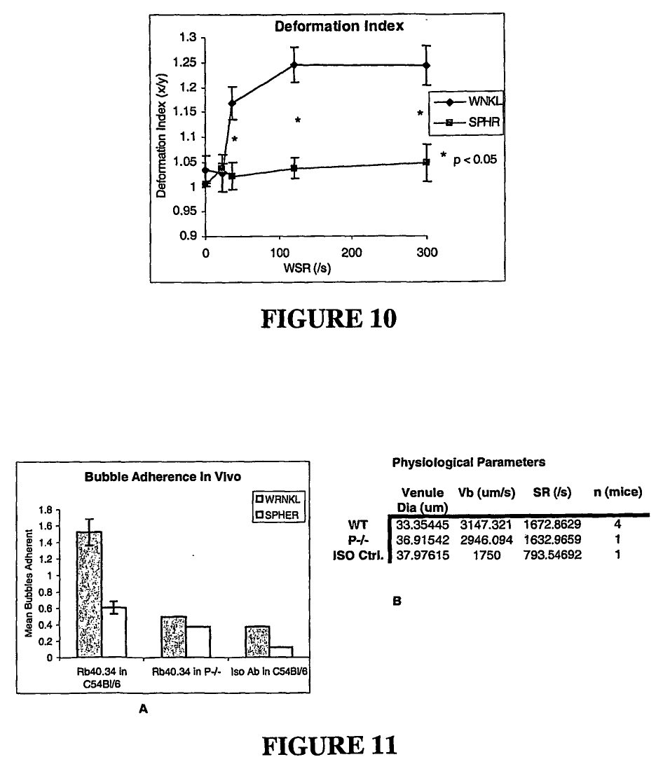

The deformability of the spherical and wrinkled microbubbles under variable shear conditions was examined to test the hypothesis that the enhanced fun attachment efficiency of the wrinkled microbubbles was facilitated by a deformation-induced increase in bubble surface area available to binding. The wrinkled microbubbles deformed significantly more than the spherical microbubbles over the range of shear at which attachment efficiency was examined, as shown in FIG. 10. A deformation index above 1 indicates elongation in the direction of flow, (suggesting that the deformation was induced by the shear force upon the bubble) and a corresponding increase in microbubble area in contact with the P-selectin.Fc substrate. A greater number of P-selectin.Fc:Rb40.34 bonds may be formed, thus stabilizing firm adhesion. However, the deformation index does not appear to correlate with the dependence of firm attachment efficiency on shear rate presented in Section 2.4.2: the deformation index increases with shear, but firm adhesion decreases with shear. This discrepancy may be due to the opposing actions of the shear force upon a bound bubble. Increasing shear rate increases the magnitude of the shear force pushing on the bubble, causing an increased deformation of its surface. However, this shear force also exerts a dislodging drag force upon the bound bubble, forcing it from the plate surface. The relative magnitudes of these two opposing effects is determined by the adherent bubble's susceptibility to shear drag and the relative ease with which its lipid shell may be deformed.

2.5 Increased Adhesion in Mouse Cremaster

Attachment efficacy of wrinkled and spherical microbubbles in vivo was examined in a mouse model of inflammation. The results are shown in FIG. 11. Cytokine-stimulated venules with shear rates between 500-3000 s⁻¹ were examined for microbubble attachment. In four c57B1/6 mice, the wrinkled microbubbles exhibited significantly greater attachment than the spherical microbubbles. Using targeted microbubbles in P-selectin deficient mice (P−/−) or targeting the microbubbles with an isotype control antibody (Iso Ab) reduced attachment approximately 2-fold for both microbubble populations. Although the spherical microbubbles are ˜0.5 μm larger in diameter than the wrinkled bubbles, it is unlikely that this size difference contributes to differential delivery to the endothelial surface, as both populations are of the appropriate size to traverse mouse capillaries. Moreover, there was no observable difference in size or elongation between adherent wrinkled and spherical bubbles. The adherent microbubbles in the P−/− mouse or in the isotype control experiment were likely attached to macrophages, as reported by Lindner and colleagues (2001).

2.6 Echogenic Response for Wrinkled and Spherical Microbubbles

The contrast provided by the crenated microbubbles was approximately equal to that of round microbubbles at equal concentration when infused into a 0.5 L bag of injection-grade 0.9% NaCl, as determined visually; moreover, echogenicity increases with bubble concentration within the range examined.

In the experiments wherein crenated or spherical (untreated) microbubbles coated with biotin as described in section 1 of the Experimental were infused over the avidin-coated dishes in the parallel plate flow chamber at a low shear rate, the ultrasound contrast signal increased with the number of bubbles bound to the dish, and again was approximately equal for equal concentrations of round and crenated bubbles. Microbubbles bound to the dish were destroyed by increasing the mechanical index of the ultrasound wave above 0.5 (after several ultrasound pulses, the signal from bubbles disappeared). This confirmed that the contrast signal observed was in fact due to adherent bubbles.

While the invention has been illustrated and described in detail in the foregoing description, the same is to be considered as illustrative and not restrictive in character, it being understood that only the preferred embodiments have been described and that all changes and modifications that come within the spirit of the invention are desired to be protected. In addition, all publications identified in this application are indicative of the abilities possessed by those of ordinary skill in the art, and are each hereby incorporated by reference as if individually incorporated by reference and fully set forth.

### REFERENCES

Forstberg F and Shi W T. 2001. Physics of contrast microbubbles. In Ultrasound Contrast Agents, 2^{nd} Edition. Ed. Boldberg B B, Raichlen J S, Forsberg F. Martin Dunitz, LTD, London, UK.
Klibanov A L, Hughes M S, Villanueva F S, Jankowski R J, Wagner W R, Wojdyla J K, Wible J H, Brandenburger G H. 1999. Targeting and ultrasound imaging of microbubble-based contrast agents. MAGMA. 8: 177-184.
Crouse L J, Cheirif J, Hanly D E. 1993. Opacification and border delineation improvement in patients with suboptimal endocardial border definition in routine echocardiography: Results of the Phase III Albunex Multicenter Trial. Journal of American Cardiology 22: 1494-1500.
Shub C, Tajik A J, Seward J B. 1976. Detecting intrapulmonary right-to-left shunts with contrast echocardiography. Mayo Clinic Proceedings 51: 81-84.
Rim S, Leong-Poi H, Lindner J R, Couture D, Ellegala D, Mason H, Durieux M, Kassel N F, Kaul S. 2001. Quantification of cerebral perfusion with “real-time” contrast-enhanced ultrasound. Circulation. 104: 2582-2587.
Mulvagh S L, DeMaria A, Feinstein S. 2000. Contrast Echocardiography: Current and future applications. Journal of the American Society of Echocardiography. 13: 331-342.
Villanueva F S. 2000. The use of myocardial contrast echocardiography in clinical evaluation after myocardial infarction. Coronary Artery Disease. 11: 235-242.
Villanueva F S, Jankowski R J, Manaugh C, Wagner W R. 1997. Albumin microbubble adherence to human coronary endothelium implications for assessment of endothelial function using myocardial contrast echocardiography. Journal of American Cardiology. 30: 689-693.
Gunda M and Mulvagh S L. 2001. Recent advances in myocardial contrast echocardiography. Current Opinion in Cardiology. 15: 231-239.
Klibanov A L, Hughs M S, Marsh J N, Hall C S, Miller J G, Wible J W, Brandenburger G H. 1997. Targeting of ultrasound contrast material: an in vitro feasibility study. Acta Radiologica. 38: 113-120.
Girard M S, Kono Y, Sirlin C B. 2001. B-mode enhancement of the liver with microbubble contrast: a blinded study in rabbits with VX2 tumors. Acadamec Radiology. 8: 734-740.
Walday P, Tolleshuag H, Gjoen T, Kindberg G M, Berg T, Skotland T, Holtz E. 1994. Biodistributions of air-filled albumin microspheres in rats and pigs. Biochemical Journal. 299: 437443.
Keller M W, Spotnitz W D, Metthew T L, Glasheen W P, Watson D D, Kaul S. 1990. Intraoperative assessment of regional myocardial perfusion using quantitative myocardial contrast echocardiography: an experimental evaluation. Journal of American Colleges of Cardiology. 16: 1267-1279.
Kono, Y, Steinbach, G C, Peterson T, Schmid-Schonbein G W, Mattrey R P. 2002. Mechanism of parenchymal enhancement of the liver with a microbubble-based US contrast medium: an intravital microscopy study in rats. Radiology. 224: 253-257.
Leong-Poi H, Christiansen J, Klibanov A L, Kaul S, Lindner J R. 2003. Noninvasive assessment of angiogenesis by ultrasound and microbubbles targeted to αᵥ-integrins. Circulation. 107: 455-460.
Lindner J R, Coggins M P, Kaul S, Klibanov A L, Brandenburger G H, Ley K. 2000. Microbubble persistence in the microcirculation during ischemia/reperfusion and inflammation is caused by integrin- and complement-mediated adherence to activated leukocytes. Circulation. 101: 668-675.
Lindner J R, Dayton P A, Coggins M P, Ley K, Song J, Ferrara K, Kaul S. 2000. Noninvasive imaging of inflammation by ultrasound detection of phagocytosed microbubbles. Circulation. 102: 531-538.
Lindner J R, Ismail S, Spotnitz W D, Skyba D M, Jayaweera A R, Kaul S. 1998. Albumin microbubble persistence during myocardial contrast echocardiography is associated with microvascular endothelial glycocalyx damage. Circulation. 98: 2187-2194.
Lindner J R, Song J, Christiansen, J, Klibanov, A L, Xu, F, Ley, K. 2001. Ultrasound assessment of inflammation and renal tissue injury with microbubbles targeted to P-selectin. Circulation. 104: 2107-2112.
Lindner J R, Song J, Xu F, Klibanov A L, Singbartl K, Ley K, Kaul S. 2000. Noninvasive ultrasound imaging of inflammation using microbubbles targeted to activated leukocytes. Circulation. 102: 2745-2750.
Christiansen J P, Leong-Poi H, Klibanov A L, Kaul S, Lindner J R. 2002. Noninvasive imaging of myocardial reperfusion injury using leukocyte-targeted contrast echocardiography. Circulation. 105: 1764-1767.
Ellegala D B, Leong-Poi H, Carpenter J E, Klibanov A L, Kaul S, Shaffrey M E, Sklenar J, Lindner J R. 2003. Imaging tumor angiogenesis with contrast ultrasound and microbubbles targeted to αᵥβ₃. Circulation. 108: 336-341.
Schumann P A, Christiansen J P, Quigley R M, McCreery T P, Sweitzer R H, Unger E C, Lindner J R, Matsunaga T O. 2003. Targeted-microbubble binding selectively to GPIIb/IIIa receptors of platelet thrombi. Investigative Radiology. 37: 587-593.
Price R J, Skyba D M, Kaul S, Skalak T C. 1998. Delivery of colloidal particles and red blood cells to tissue through microvessel ruptures created by targeted microbubble destruction with ultrasound. Circulation 98: 1264-1267.
Teupe C, Richter S, Fisslthaler B, Randriamboavonjy V, Ihling C, Fleming I, Busse R, Zeiher A M, Dimmeler S. 2002. Vascular gene transfer of phosphomimetic endothelial nitric oxide synthase (S1177D) using ultrasound-enhanced destruction of plasmid-loaded microbubbles improves vasoreactivity. Circulation 105: 1105-1109.
Porter T R, Hiser W L, Kricsfeld D, Deligonul U, Xie F, Iversen P, Radio S. 2001. Inhibition of carotid artery neointimal formation with intravenous microbubbles. Ultrasound in Medicine and Biology. 27:259:265.
Weller G E R, Lu E, Csikari M M, Klibanov A L, Fischer D, Wagner W R, Villanueva F S. 2003. Ultrasound imaging of acute cardiac transplant rejection with microbubbles targeted to intercellular adhesion molecule-1. Circulation. 108: 218-224.
Finger E B, Bruehl R E, Bainton D F, Springer T A. 1996. A differential role for cell shape in neutrophil tethering and rolling on endothelial selectins under flow. The Journal of Immunology 157: 5085-5096.
Atherton A and Born G V R. 1972. Quantitative investigation of the adhesiveness of circulating polymorphonuclear leukocytes to blood vessel walls. The Journal of Physiology 222: 447-474.
Firrell J C and Lipowsky H H. 1989. Leukocyte margination and deformation in mesenteric venules of rat. American Journal of Physiology 256: H1667-H1674. Forstberg F and Shi W T. 2001. Physics of contrast microbubbles. In Ultrasound Contrast Agents, 2^{nd} Edition. Ed. Boldberg B B, Raichlen J S, Forsberg F. Martin Dunitz, LTD, London, UK.
Park E Y H, Smith M J, Stropp E S, Snapp K R, DiVietro J A, Walker W F, Schmidtke D W, Diamond S L, Lawrence M B. 2002. Comparison of PSGL1 microbead and neutrophil rolling: microvillus elongation stabilizes P-selectin bond clusters. Biophysical Journal 82: 1835-1847.
Yago T, Leppänen A, Qiu H, Marcus W D, Nollert M U, Zhu C, Cummings R D, McEver, R P. 2002. Distinct molecular and cellular contributions to stabilizing selectin-mediated rolling under flow. Journal of Cell Biology. 158: 787-799.
Olsen T S, Singbartl K, Ley K. 2001. L-selectin is required for fMLP-but not C5a-induced margination of neutrophils in pulmonary circulation. American Journal of Physiology 282: R1245-R1242.
Kim D H, Klibanov A L, Needham D. 2000. The influence of tiered layers of surface-grafted poly(ethylene glycol) on receptor-liband-mediated adhesion between phospholipid monolayer-stabilized microbubbles and coated blass beads. Langmuir. 16:2804-2817.
Green, N. M. 1965. A spectrophotometric assay for avidin and biotin based on binding of dyes by avidin. Biochem. J. 94:23c-24c.
Reneman R S, Woldhuis B, Oude Egbrink M G A, Slaaf D W, Tangelder G J. 1992. Concentration and velocity profiles of blood cells in the microcirculation. Anonymous Advances in Cardiovascular Engineering p2540.

US patent Documents

| | | |
|---|---|---|
| 6,331,289 | December, 2001 | Klaveness et al |
| 6,245,318 | June 2001 | Klibanov et al |
| 6,264,917 | July 2001 | Klaveness et al |
| 6,416,740 | July 2002 | Unger |
| 6,443,898 | Sept 2002 | Unger et al |

1. A microbubble composition for binding to a target, comprising:
gas-filled microbubbles in a liquid carrier;
said microbubbles substantially having crenated microbubble membranes; and said membranes including binding targeting molecules that bind to the target.

2. The microbubble composition of claim 1, wherein the microbubble membranes comprise a lipid, protein, polymer or other surfactant, or a combination thereof.

3. The microbubble composition of claim 1, wherein the gas is substantially insoluble in blood.

4. The microbubble composition of claim 3, wherein the gas is a fluorine-containing gas.

5. The microbubble composition of claim 1, wherein the microbubbles have a mean diameter of about 1 to about 10 micrometers.

6. The microbubble composition of claim 1, wherein the target is a receptor, and wherein the binding targeting molecules bind to the receptor.

7. The microbubble composition of claim 6, wherein the receptor is selected from the group consisting of extracellular matrix proteins, adhesion molecules, G-protein coupled receptors, cell surface proteins, cytokines, glycoproteins, peptides, lipids, glycolipids, carbohydrates or combinations thereof.

8. The microbubble composition of claim 1, wherein the targeting molecules are selected from the group consisting of peptides, peptide mimetics, aptamers, proteins, antibodies and antibody fragments, oligosaccharides, and small organic molecules.

9. A microbubble composition useful for binding to a target, comprising:
a suspension of gas-filled microbubbles in a liquid carrier, said microbubbles substantially having microbubble membranes having surface projections, said membranes further including binding targeting molecules that bind to the target.

10. The microbubble composition according to claim 9, wherein said surface projections comprise membrane folds.

11. The microbubble composition of claim 9, wherein the membranes comprise a lipid, protein or surfactant, and wherein the microbubbles have a mean diameter of about 1 to about 10 micrometers.

12. The microbubble composition of claim 9, wherein the gas is substantially insoluble in blood.

13. The microbubble composition of claim 12, wherein the target is a cell membrane bound receptor, and wherein the targeting molecules bind to the receptor.

14. The microbubble composition of claim 9, wherein the targeting molecules are selected from the group consisting of peptides, peptide mimetics, aptamers, proteins, antibodies and antibody fragments, oligosaccharides, and small organic molecules.

15. The microbubble composition of claim 13, wherein the receptor is selected from the group consisting of extracellular matrix proteins, adhesion molecules, G-protein coupled receptors, cell surface proteins, cytokines, glycoproteins, peptides, lipids, glycolipids, carbohydrates or combinations thereof.

16. A microbubble composition useful for binding to a target, comprising:
a suspension of microbubbles in a liquid carrier, said microbubbles predominantly having non-spherical microbubble membranes, said non-spherical microbubble membranes exhibiting increased deformability under shear relative to corresponding spherical microbubble membranes, and said microbubble membranes comprising a binding targeting molecule for binding to the target.

17. The microbubble composition of claim 16, wherein the membranes comprise a lipid, protein, polymer or other surfactant, or a combination thereof.

18. The microbubble composition of claim 16, wherein said gas is substantially insoluble in blood.

19. The microbubble composition of claim 16, wherein the microbubbles have a mean diameter of about 1 to about 10 micrometers.

20. The microbubble composition of claim 16, wherein the target is a cell membrane bound receptor, and wherein the targeting molecules bind to the receptor.

21. A method for binding microbubbles to a target, comprising:
contacting the target with a microbubble composition according to claim 1.

22. A method according to claim 21, wherein microbubble membranes of the microbubble composition include a targeting molecule attached by a spacer arm.

23. A method for preparing a targeted microbubble composition, comprising:
forming gas-filled microbubbles having spherical microbubble membranes suspended in a liquid carrier;
converting the spherical microbubble membranes to non-spherical microbubble membranes; and
attaching to or incorporating into said microbubble membranes targeting molecules for binding to a target.

24. The method of claim 23, wherein said targeting molecules are attached to or incorporated into the membranes prior to said converting.

25. The method of claim 23, wherein said targeting molecules are attached to or incorporated into the membranes after said converting.

26. The method of claim 23, wherein said converting includes causing a partial release of gas from within the spherical microbubble membranes.

27. The method of claim 26, wherein said converting includes subjecting the spherical microbubble membranes to pressure.

28. The method of claim 27, wherein said pressure is applied by hydrostatic pressure, ultrasonic waves, or an osmotic pressure gradient across the microbubble membrane.

29. The method of claim 23, wherein the targeting molecules are selected from the group consisting of peptides, peptide mimetics, aptamers, proteins, antibodies and antibody fragments, oligosaccharides, and small organic molecules.

30. A pharmaceutical composition, comprising a microbubble composition according to claim 1, wherein the liquid carrier is a pharmaceutically acceptable liquid carrier.

31. A pharmaceutical composition according to claim 30, which is a therapeutic composition.

32. A pharmaceutical composition according to claim 30, which is a diagnostic composition.

33. A pharmaceutical composition according to claim 32, which is an ultrasound contrast agent.

34. A method for ultrasound imaging in a patient, comprising:
introducing into the patient an ultrasound contrast agent according to claim 33; and
developing an ultrasound image based upon said composition.

35. A method for therapeutic treatment of a patient, comprising administering to the patient a therapeutic composition according to claim 31.
